(19) United States
(12) Patent Application Publication
(10) Pub. No.: US 2017/0027721 A1
(43) Pub. Date: Feb. 2, 2017

(54) MEDICAL DRAINAGE DEVICES AND METHODS OF USE

(71) Applicant: Boston Scientific Scimed, Inc., Maple Grove, MN (US)

(72) Inventor: John Allen HINGSTON, Framingham, MA (US)

(73) Assignee: Boston Scientific Scimed, Inc., Maple Grove, MN (US)

(21) Appl. No.: 15/193,731

(22) Filed: Jun. 27, 2016

Related U.S. Application Data
(60) Provisional application No. 62/198,205, filed on Jul. 29, 2015.

(51) Int. Cl. A61F 2/90; A61M 39/10; A61F 2/852; A61M 27/00

(52) U.S. Cl. CPC A61F 2/90; A61M 27/002; A61M 39/10; A61F 2/852; A61F 2210/009; A61F 2220/0025

(57) ABSTRACT

The present disclosure is directed to systems and methods for creating a drain within a duct of a patient's pancreatico-biliary system. The method may include positioning a first elongate member distally of a stricture within the duct, wherein a magnetic portion is disposed at or near a proximal end of the first elongate member. A second elongate member is positioned proximally of the stricture within the duct, wherein a magnetic portion is disposed at or near a distal end of the second elongate member. The proximal end of the first elongate member is spaced apart from the distal end of the second elongate member, and the magnetic portion of the first elongate member and the magnetic portion of the second elongate member are attracted to each other so that at least one of the first elongate member and the second elongate member is pulled into the stricture within the duct.

### CROSS-REFERENCE TO RELATED APPLICATIONS

This application claims the benefits of priority from U.S. Provisional Application No. 62/198,205, filed on Jul. 29, 2015, the entirety of which is incorporated herein by reference.

### TECHNICAL FIELD

Aspects of the present disclosure relate generally to medical devices. More particularly, aspects of the disclosure relate to devices for providing drainage in areas within a patient's body to, for example, treat strictures within the pancreatico-biliary, urinary, and/or vascular systems. Aspects of the disclosure also cover methods of using such devices.

### BACKGROUND OF THE DISCLOSURE

Various medical conditions, disorders, and dysfunctions may be treated by performing a medical intervention to provide drainage. One example of such a condition is a stricture formed within a duct or tract in a patient's pancreatico-biliary, urinary, or vascular system. Such a stricture can slow or even stop fluid flow through the duct or tract. A conventional method for treating a stricture includes introducing a stent into the affected duct or tract. There may be drawbacks associated with using traditional stents, including, but not limited to, stent migration, re-occlusion of the duct or tract due to tissue ingrowth, and/or the need for re-intervention for replacing stents.

### SUMMARY OF THE DISCLOSURE

Aspects of the present disclosure provide systems, devices, and methods for performing various medical procedures, including creating drainage for blocked body lumens, such as strictured ducts or tracts within the pancreatico-biliary, urinary, and/or vascular systems.

According to one aspect of the present disclosure, a medical device for creating a drain within a lumen of a patient's body, may include a first elongate member including a proximal end, a distal end, a lumen extending between the proximal end and distal end, and a first magnetic portion disposed at or near the proximal end and surrounding the lumen. The medical device may also include a second elongate member including a proximal end, a distal end, a lumen extending between the proximal end and distal end, and a second magnetic portion disposed at or near the distal end of the second elongate member and surrounding the lumen of the second elongate member. The proximal end of the first elongate member may be configured to be drawn toward the distal end of the second elongate member by magnetic attraction between the first and second magnetic portions.

Additionally or alternatively, the medical device may include one or more other features describe here. At least one opening may be formed in a surface of the first magnetic portion. In another example, at least one opening may be formed in a surface of the second magnetic portion. Each of the first elongate member and the second elongate member may include a tubular plastic member. The first magnetic portion may include a magnet embedded in the first elongate member. The first elongate member may include an anchor extending radially outward from an outer surface of the first elongate member. The anchor may include a base at the outer surface of the first elongate member, and a tip, the tip terminating at a point. The tip may be distal to the base. At least one of the first elongate member and the second elongate member may have an outer diameter between approximately 1 mm and approximately 15 mm, or between approximately 2 mm and approximately 10 mm in diameter. At least one of the first elongate member and the second elongate member may have a length between approximately 15 mm and approximately 200 mm, or between approximately 40 mm and 180 mm. Each of the first elongate member and the second elongate member may be at least partially formed by a stent. The first magnetic portion may include one magnet. Alternatively, the first magnetic portion may include a plurality of magnets. For example, the first magnetic portion may include eight magnets or any other suitable quantity of magnets. The first magnetic portion may be mechanically fixed to one or more wires of one of the metal wire stents. When the first magnetic portion connects to the second magnetic portion, longitudinal axes of the first elongate member and the second elongate member may be aligned. When the first magnetic portion connects to the second magnetic portion, the lumens of the first elongate member and the second elongate member may form a fluid passage extending from the distal end of the first elongate member to the proximal end of the second elongate member.

According to another aspect of the present disclosure, a method of creating a drain within a lumen of a patient's body may include positioning a first elongate member distally of a blockage in the lumen, wherein at least one magnetic portion is disposed at or near a proximal end of the first elongate member. The method may also include positioning a second elongate member proximally of the blockage in the lumen. At least one magnetic portion may be disposed at or near a distal end of the second elongate member. A gap may initially exists between the proximal end of the first elongate member and the distal end of the second elongate member. Attraction between the at least one magnetic portion of the first elongate member and the at least one magnetic portion of the second elongate member may facilitate a reduction in the size of the gap.

Additionally or alternatively, the method may include one or more other features described here. Exerting pressure on tissue forming the blockage with the proximal end of the first elongate member and the distal end of the second elongate member via the magnetic attraction. Causing pressure necrosis of the tissue via the exerted pressure. Forming the drain through connection of the proximal end of the first elongate member with the distal end of the second elongate member. Each of the first elongate member and the second elongate member may include a tubular plastic member. At least one magnetic portion of the first elongate member may be a single ring-shaped magnet. At least one magnetic portion of the first elongate member may include a recess. Each of the first elongate member and the second elongate member may be at least partially formed by a stent. At least one magnetic portion of the first elongate member may include at least eight discrete magnets.

According to another aspect of the present disclosure, a method of creating a drain within a duct of a patient's pancreatico-biliary system may include positioning a first elongate member distally of a stricture within the duct, wherein at least one magnetic portion may be disposed at or near a proximal end of the first elongate member. The method may also include positioning a second elongate member proximally of the stricture within the duct. At least one magnetic portion may be disposed at or near a distal end of the second elongate member. The proximal end of the first elongate member may be spaced apart from the distal end of the second elongate member. The at least one magnetic portion of the first elongate member and the at least one magnetic portion of the second elongate member may be in close enough of a proximity to each other that magnetic attraction therebetween pulls at least one of the first elongate member and the second elongate member into the stricture within the duct.

Additionally or alternatively, the method may include one or more other features described here. Exerting pressure on tissue forming the stricture with the proximal end of the first elongate member and the distal end of the second elongate member via the magnetic attraction. Causing pressure necrosis of the tissue via the exerted pressure. Forming the drain through connection of the proximal end of the first elongate member with the distal end of the second elongate member. Engaging the duct with at least one anchor extending radially outwardly form an outer surface of at least one of the first elongate member and the second elongate member. Elongating at least one of the first elongate member and the second elongate member via the magnetic attraction between the magnetic portions

Additional objects and advantages of the disclosed aspects will be set forth in part in the description that follows, and in part will be apparent from the description, or may be learned by practice of the disclosed aspects. The objects and advantages of the disclosed aspects will be realized and attained by means of the elements and combinations particularly pointed out in the appended claims.

It is to be understood that both the foregoing general description and the following detailed description are exemplary and explanatory only and are not restrictive of the scope of disclosed aspects, as set forth by the claims.

### BRIEF DESCRIPTION OF THE DRAWINGS

The accompanying drawings, which are incorporated in and constitute a part of this specification, illustrate various exemplary aspects and together with the description, serve to explain the principles of the disclosed aspects.

FIG. 1 is a perspective view of an exemplary two-part medical drainage device, according to aspects of the present disclosure;

FIG. 2 is a perspective view of another exemplary two-part medical drainage device, according to aspects of the present disclosure; and

FIG. 3 is a partial cut-away view of an area of a patient's body, showing an endoscope positioning a two-part medical drainage device at a target area, according to aspects of the present disclosure.

### DETAILED DESCRIPTION

Reference will now be made in detail to exemplary aspects of the disclosure, examples of which are illustrated in the accompanying drawings. Wherever possible, the same reference numbers will be used throughout the drawings to refer to the same or like parts. The term “distal” refers to a position farther away from an operator end of the device. The term “proximal” refers a position closer to the operator end of the device. As used herein, the term “approximately” indicates a range of values within +/−5% of a stated value.

Aspects of the present disclosure relate to systems, devices, and methods for providing drainage to various body parts including, e.g., the pancreatico-biliary, urinary, and vascular systems. The medical drainage device described herein is a two-part drainage device. More specifically, in exemplary aspects, one part of the medical drainage device is configured to be placed on a distal side of a blockage (e.g., a stricture within the pancreatico-biliary tree), and the other part is configured to be placed on the proximal side of the blockage. Each part of the two-part device may include magnets configured to pull the two parts together while applying pressure to sections of the stricture disposed therebetween. Without being bound by theory, it is believed that over time, pressure necrosis may kill the tissue cells of the section of the stricture between the two parts and, eventually, the two parts may connect to form a single drain to allow the free passage of biliary duct fluids. In some examples, this two-part device may include two tubes. In other examples, the two-part device may include two stents.

Additional variations are also contemplated, as described below. Each of these examples described herein may include one or more of the features described in connection with any of the other disclosed examples.

I. Tubular Drainage Device

A. Tubes

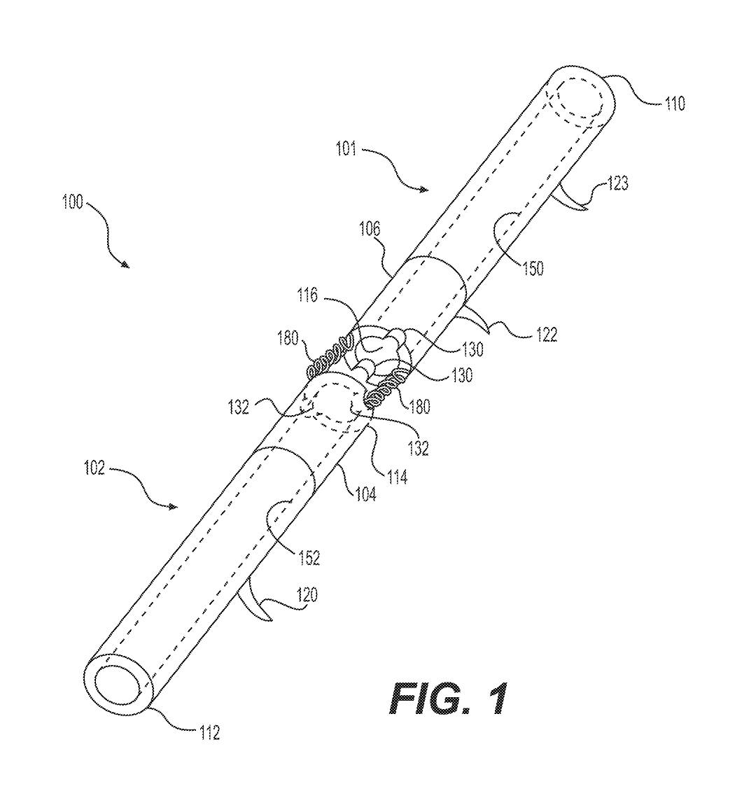

FIG. 1 illustrates exemplary aspects of a medical drainage device 100. Medical device 100 may include a distal tube 101 and a proximal tube 102. Tubes 101 and 102 may be hollow, flexible, and/or elongate tubes. Distal tube 101 has a distal end 110 and a proximal end 116, and defines a lumen 150 extending therethrough. Proximal tube 102 has a distal end 114 and a proximal end 112, and defines a lumen 152 extending therethrough. Tubes 101 and 102 are configured so that when in use in a patient's body, distal end 114 of proximal tube 102 and proximal end 116 of tube 101 may face each other. As such, distal end 114 and proximal end 116 are referred to herein as the facing ends of medical device 100, or the facing ends of tubes 101 and 102. The facing ends may extend in a direction perpendicular to the longitudinal axes of their respective tubes 101 and 102. Alternatively, the facing ends may extend at non-perpendicular angles relative to the longitudinal axes. It is also contemplated that the distal end 114 and proximal end 116 may be complementary regardless of the configuration of the facing ends.

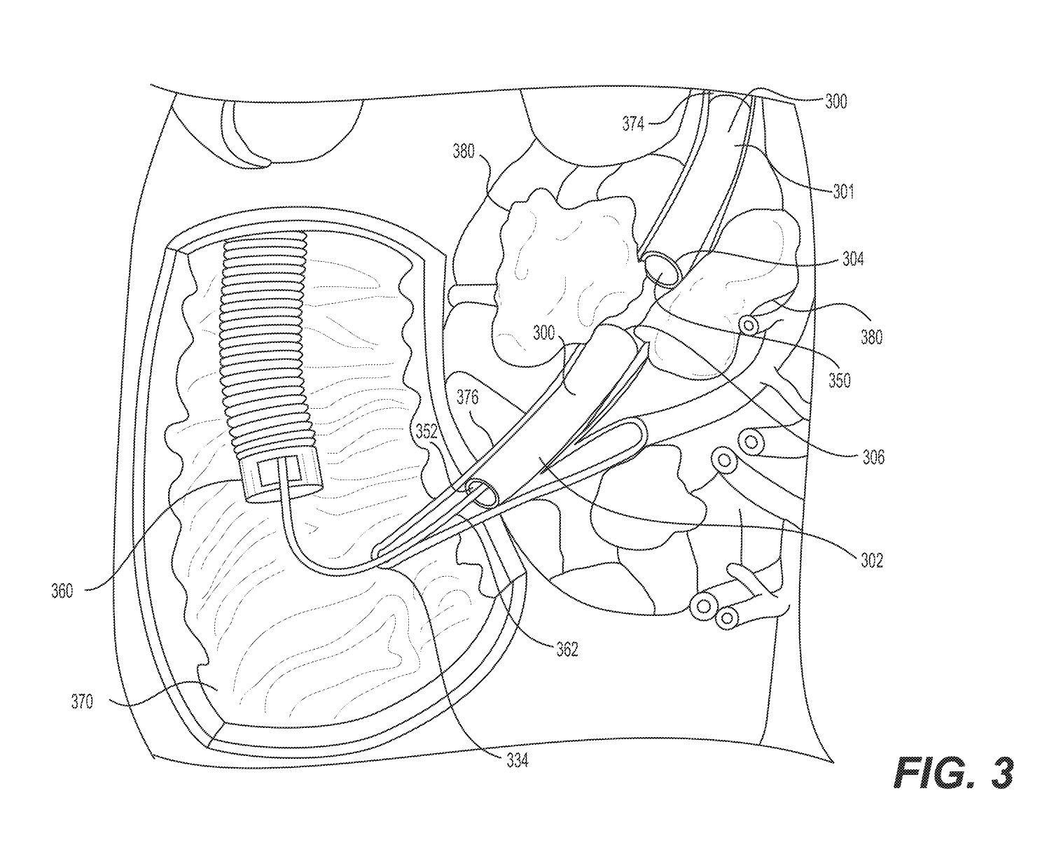

Cross-sections of tubes 101 and 102 may be circular, elliptical, irregular, and/or any shape suitable for entering a patient's body, pre-loading onto or into a positioning catheter (e.g., a catheter 362 in FIG. 3), and/or pushing past a stricture (e.g., a stricture 380 in FIG. 3). Tubes 101 and 102 may have a uniform shape along their lengths, or may have a varying shape, such as a taper at one or more ends to facilitate insertion and positioning within the body. Depending upon the particular implementation and environment of use, the length of tubes 101 and 102 may vary. For example, in implementations in which drainage device 100 is placed within the pancreatico-biliary system, each of tubes 101 and 102 may be between approximately 15 mm and approximately 200 mm long, or between approximately 40 mm and approximately 180 mm long. The diameter of tubes 101 and 102 may be tailored based on the dimensions of a body cavity or lumen. For example, each of tubes 101 and 102 may have an outer diameter between approximately 1 mm and approximately 15 mm, between approximately 2 mm and approximately 10 mm, between approximately 2.3 mm and 3.3. mm, or approximately 3 mm. The wall thickness of each of tubes 101 and 102 may be between approximately 0.1 mm and approximately 1 mm, or approximately 0.5 mm. In some implementations, tubes 101 and 102 may have the same lengths, same diameters, and/or same shapes. In some implementations, one or more of length, diameter, and shape may differ between tubes 101 and 102.

Tubes 101 and 102 may be designed to reduce the risk of damaging the surrounding tissues while in use. To this end, one or more portions of tube 101 and/or tube 102 may include one or more atraumatic geometrical structures, such as rounded or beveled terminal ends or faces (not shown), to reduce trauma and/or irritation to surrounding tissues.

To facilitate the position of tubes 101 and 102 within a patient's body, and/or to track movement of tubes 101 and 102 once positioned within the patient's body, the operator may want to know the location, position, and/or orientation of tubes 101 and 102 in the patient's body. To this end, one or more portions of tubes 101 and 102 may be radiopaque, such as by inclusion of barium sulfate in plastic material used to form tubes 101 and 102, or inclusion of one or more metal portions on or in tubes 101 and 102, configured to provide sufficient radiopacity. These inclusions may facilitate detection of a location, position, and/or orientation of tubes 101 and 102 within a patient's body. An operator, with the aid of suitable imaging equipment, may monitor tubes 101 and 102 as they are positioned, and/or as they move toward each other when in use. In one example, the facing ends of tubes 101 and 102 may be radiopaque. Such a configuration may provide an operator with an indication of proper positioning and desired movement, used on, for example, the orientation of the facing ends relative to each other.

Tubes 101 and 102 may be rigid along their entire length, flexible along their entire length, flexible along a portion of their length, or configured for flexure at only predetermined locations. In one example, the flexibility of tubes 101 and 102 may allow them to be steered through body lumens and placed into position on both sides of a stricture. For example, a catheter/endoscope assembly (such as a catheter 362/endoscope 360 described below with respect to FIG. 3) may include a steering system (not shown) to deflect the catheter/endoscope and, as a result, move at least a portion of tubes 101 and 102 up/down and/or side-to-side.

Tubes 101 and 102 may be formed of any suitable material capable of traveling through the patient's body and providing a drainage lumen through a blockage, e.g., a stricture in a duct or tract. In general, tubes 101 and 102 may be made of any suitable material that is biocompatible. That is, tubes 101 and 102 may be non-toxic and/or non-injurious to living tissue, and use of materials that may cause immunological reactions or rejections may be avoided. In some examples, tubes 101 and 102 may be made of polymeric elastomers, rubber tubing, and/or medically approved polyvinylchloride tubing. Polymeric elastomers may include, for example, EVA (ethylene vinyl acetate), silicone, polyurethane, and/or a biopharmaceutical tubing made of a thermoplastic elastomer, such as C-FLEX. Tubes 101 and 102 may be capable of expanding after insertion into the desired position. In some implementations tubes 101 and 102 may be made of a shape memory material, e.g., a superelastic material such as nitinol.

B. Magnets

A magnetic portion 106 may be disposed at or near proximal end 116 of distal tube 101. A magnetic portion 104 may be disposed at or near distal end 114 of proximal tube 102. In some examples, magnetic portions 104 and 106 may be affixed to the facing end of tubes 101 and 102 and/or embedded partially or completely within tubes 101 and 102. In some examples, tubes 101/102 and magnetic portions 104/106 may be formed by overmolding, which may involve placing magnetic portions 104/106 into an injection mold and injecting material around at least a portion of magnetic portions 104/106.

Magnetic portions 104 and 106 may include any suitable, known magnets. Magnetic portions 104 and 106 may include rare-earth magnets such as, but not limited to, ones made of samarium-cobalt and neodymium. Magnetic portions 104 and 106 may be a sufficient strength to, over time, cause pressure necrosis of tissue of at a stricture. For example, the attractive force between magnetic portions 104 and 106 may cause the facing ends of tube 101 and 102 to forcibly engage the tissue, leaving to pressure necrosis of the tissue. Magnetic portions 104 and 106 may be any shape, including, for example, ring-shaped. In some examples, magnetic portions 104 and 106 may form the facing ends of tubes 102 and 101 (e.g., distal end 114 and proximal end 116, respectively). Magnetic portions 104 and 106 may be between approximately 4 mm and 8 mm in length, or between approximately 0.5 mm and approximately 20 mm in length. Magnetic portions 104 and 106 may be configured to have the same orientation of polarity, e.g., the distal-facing sides of magnetic portions 104 and 106 may be N-poles while the proximal-facing sides of magnetic portions 104 and 106 may be S-poles. In such a configuration, the facing ends of tubes 101 and 102 will have opposite polarity and thus will be attracted to each other. For example, the distal-facing side of magnetic portion 104 (e.g., the distal end of tube 102) may be a N-pole, while the proximal-facing side of magnetic portion 106 (e.g., the proximal end of tube 101) may be a S-pole.

Additionally or alternatively, medical device 100 may include one or more tethers (e.g., tethers 180 of FIG. 1) for tethering tubes 101 and 102 to each other. In some examples, tethers 180 may be elastic. Elastic tethers 180 may help pull tubes 101 and 102 together. For example, elastic tethers 180 may be stretched when tubes 101 and 102 are placed on both sides of the stricture, with elastic tethers 180 extending through the stricture. As elastic tethers 180 return back to a rest state, tubes 101 and 102 may be drawn towards each other. In implementations with elastic tethers 180, magnets may be omitted, or weaker magnets may be used, to pull tubes 101 and 102 together.

C. Anti-Migration/Anchoring Features

After tubes 101 and 102 are placed on opposite sides of a stricture, the two components may be drawn closer together due to the attraction between magnetic portions 104 and 106. While the two components move closer together, and/or after they connect, movement of the two components away from the stricture should be avoided to ensure that the drain formed by the two components remains at the stricture. Movement away from the stricture may result in the re-forming of an occlusion at the stricture. In order to prevent medical device 100 from migrating away from the stricture, medical device 100 may incorporate anti-migration and/or anchoring features. Medical device 100 may include any suitable anti-migration and/or anchoring device(s) known in the art. Through incorporation of such anti-migration and/or anchoring features, proximal tube 102 may be configured to limit its own proximal movement away from the stricture and/or distal tube 102 may be configured to limit its own distal movement away from the stricture. In one implementation, proximal tube 101, and distal tube 102 may include anchors 120 and 122, respectively. In examples in which tubes 101 and 102 are made of, for example, plastic or metal, anchors 120 and 122 may be formed from and sliced out of the tubes themselves.

In one example, anchor 122 of distal tube 101 may be sliced by any sharp device capable of severing material, e.g., a blade. The slice may begin at any point along tube 101 and move proximally, proceeding deeper into the wall of tube 101 as the cut continues proximally, forming anchor 122 (e.g., a prong, tine, or barb) from the cut portion of the wall of tube 101. Such an anchor configuration, with a base of anchor 122 being proximal to a tip of anchor 122, may help secure tube 101 to tissue by, for example, limiting distal movement of tube 101, while allowing proximal movement.

Anchor 120 of proximal tube 102 may also be sliced out of the material of tube 102. The slice may begin at any point along tube 102 and move distally, proceeding deeper into the wall of tube 102 as the cut continues distally. Such an anchor configuration, with a base of anchor 120 being distal to a tip of anchor 120, may help secure tube 102 to tissue by for example, limiting proximal movement of tube 102, while allowing distal movement of tube 102 at the urging of magnetic portions 104 and 106.

In some examples, proximal tube 101 and/or distal tube 102 may include multiple anchors, configured to limit both proximal and distal movement. For example, distal tube 102 may include a second anchor 123, with its base distal to its tip, for limiting proximal movement of tube 101. It is also contemplated that anchor 123 may be provided on tube 101 and anchor 122 may be omitted to allow distal movement of tube 101. It is further contemplated that the position of anchors 122 and 123 along the length of tube 101 may be switched.

In an example in which tubes 101 and 102 are made of nitinol, anchors 120, 122, and/or 123 may also be made of nitinol. Anchors 120, 122, and/or 123 may have a contracted state, in which they are flush with the outer surfaces of tubes 101 and 102, and an expanded state, in which they project radially outward from the outer surfaces of tubes 101 and 102. Anchors 120, 122, and/or 123 may be in the contracted state during delivery and/or positioning. Anchors 120, 122, and/or 123 may expand when in position on both sides of a stricture. Expansion may be due to inherent outward biasing, deformation due to body heat, or the like.

Anchors 120, 122, and/or 123 may be between approximately 0.5 mm and 5 mm long, or between approximately 1 mm and approximately 3 mm long. The tips of anchors 120, 122, and/or 123 may be thinner than the bases. The bases may be approximately 0.5 mm to approximately 3 mm in width, or between approximately 1 mm and approximately 2 mm.

The tips of anchors 120, 122, and/or 123 may come to a point. In some examples, the tips of anchors 120, 122, and/or 123 may be sufficiently sharp to penetrate tissue, and thereby secure tubes 101 and 102 at a desired position in a patient's body lumen, e.g., in a biliary duct. Tubes 101 and 102 may have any number of anchors. For example, tubes 101 and 102 may have four anchors. In some examples, only one of tubes 101 and 102 may have any anchors. In some examples, tube 101 may have a different number of anchors than tube 102. It is also contemplated that tubes 101 and 102 may have no anchors.

Additionally or alternatively, the facing ends of tubes 101 and 102 and/or magnetic portions 104 and 106 may include pockets 130 and 132. Pockets 130 and 132 may include recesses and/or openings that allow tissue ingrowth. Tissue ingrowth may limit or prevent migration of tubes 101 and 102. In some examples, one to four pockets may be milled into one or both of magnets 104 and/or 106. As shown in FIG. 1, two pockets 132 may be milled into the distal-facing surface of magnetic portion 104 and two pockets 130 may be milled into the proximal-facing surface of magnetic portion 106. Pockets 130 and pockets 132 may or may not be aligned once tubes 101 and 102 connect. In examples in which magnets are embedded into the plastic of tubes 101 and/or 102, pockets 130 and 132 may be milled into plastic that coats the facing ends of magnetic portions 104 and 106 and/or into magnetic portions 104 and 106 themselves. Any suitable number of pockets may be utilized.

II. Stent-Based Drainage Device

A. Stent

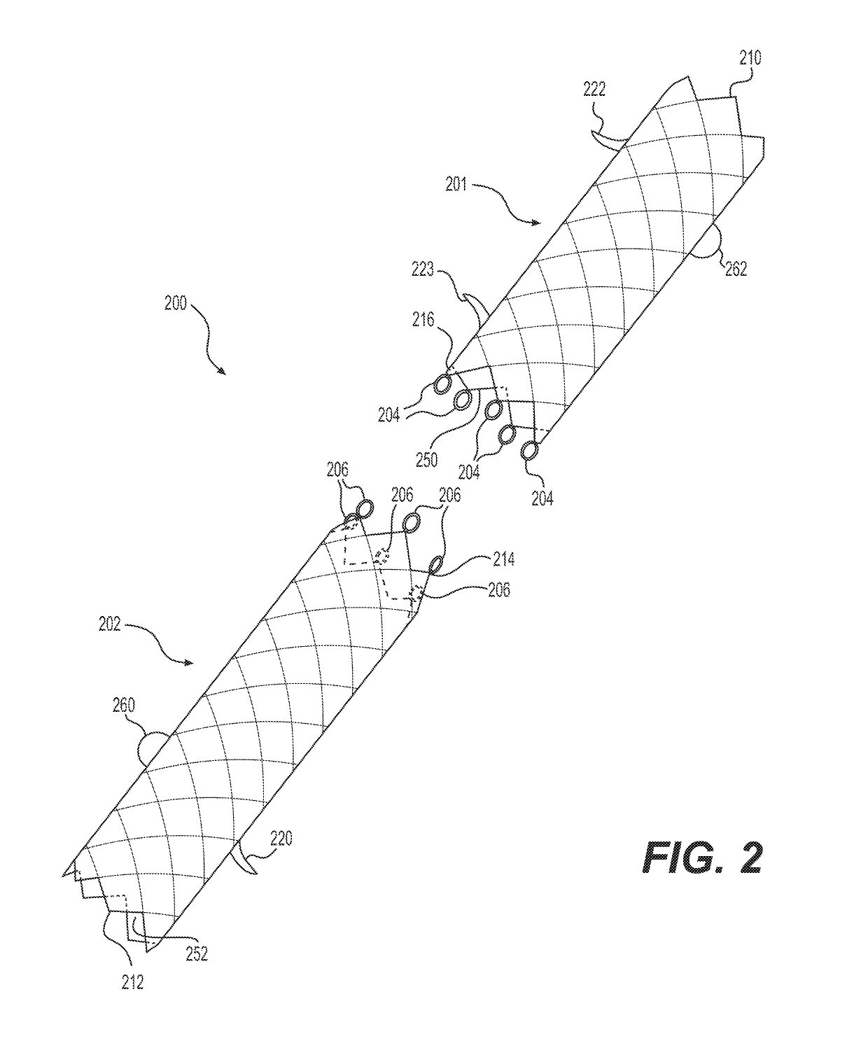

FIG. 2 illustrates exemplary aspects of a medical drainage device 200. Medical device 200 may include a distal stent 201 and a proximal stent 202. Distal stent 201 has a distal end 210 and a proximal end 216, and defines a lumen 250 extending therethrough. Proximal stent 202 has a distal end 214 and a proximal end 212, and defines a lumen 252 extending therethrough. Stents 201 and 202 are configured so that, in a patient's body, distal end 214 and proximal end 216 may face each other. Accordingly, distal end 214 and proximal end 216 are referred to herein as the facing ends of medical device 200, or the facing ends of stents 201 and 202.

The facing ends of stents 201 and 202 may extend at an angle of approximately 90 degrees relative to the longitudinal axes of stents 201 and 202. In other examples, the facing ends may extend at an angle of between approximately 30 degrees and approximately 89 degrees relative to the longitudinal axes of stents 201 and 202. In such examples, the angles of the facing ends of stents 201 and 202 may be supplemental to each other, such that distal end 214 and proximal end 216 are complementary.

Stents 201 and 202 may be configured to transition between a first collapsed configuration (e.g., for insertion) and a second expanded configuration (e.g., once in a desired position, such as in a patient's body lumen). Stents 201 and 202 may have a greater diameter in their expanded configuration than in this collapsed configuration. Stents 201 and 202 may include any suitable self-expanding mesh or coil structure. For example, stents 201 and 202 may include a braided and/or twisted lattice of wire(s), a helical or semi-helical spiral, and/or a plurality of undulating, corrugated, or sinusoidal rings. Additionally, stents 201 and 202, according to aspects of the present disclosure, may be made, at least partially, of a shape-memory material such as, for example, a Cobalt-Chromium-Nickel alloy like Elgiloy, synthetic plastics, stainless steel, and superelastic metallic alloys of Nickel and Titanium (e.g., nitinol), copper, cobalt, vanadium, chromium, iron, or the like. Alternative materials may include, but are not limited to, other metal alloys, powdered metals, ceramics, thermal plastic composites, ceramic composites, and polymers. Combinations of these and other materials may be used.

In some aspects, a thin coating or covering may extend along an outer surface of stent 201 and/or stent 202. The coating or covering may form a protective sleeve. The coating or covering may be configured to prevent stent 201 and 202 from causing unintentional damage to surrounding tissue, and/or prevent damage to stents 201 and 202 by medical instruments or patient anatomy. The coating or covering may be comprised of any suitable biocompatible polymer such as, for example, silicone. Alternatively, the coating or covering may be omitted.

Depending upon the particular implementation and environment of use, the lengths of stents 201 and 202 may vary. For example, in implementations in which drainage device 200 is placed within the pancreatico-biliary system, each of stents 201 and 202 may be between approximately 15 mm and approximately 150 mm long, or between approximately 40 mm and approximately 100 mm long. The diameters of stents 201 and 202 may be tailored based on patient anatomy, such as the dimensions of a body cavity. For example, stents 201 and 202 may have an outer diameter in the expanded configuration between approximately 5 mm and approximately 15 mm, between approximately 8 mm and approximately 10 mm, or approximately 9 mm. In some implementations, stents 201 and 202 may have the same length, same diameter, and/or same shape. In some implementations, one or more of length, diameter, and shape may differ between stents 201 and 202. It is also contemplated that stent 201 and 202 may have dimensions similar to tubes 101 and 102.

To facilitate the positioning of stents 201 and 202 within a body cavity, and/or to track their movement once positioned, the operator may want to know the location, position, and/or orientation of stents 201 and 202 in the patient's body. To this end, one or more portions of stent 201 and/or stent 202 may be radiopaque, such as by inclusion of barium sulfate in material used to form the mesh or coil structure of one of, or both of, stents 201 and 202; or by inclusion of one or more metal portions, configured to provide sufficient radiopacity. These inclusions may facilitate detection of positions and/or orientations of stents 201 and 202 within a patient's body. An operator, with the aid of suitable imaging equipment, may monitor stents 201 and 202 as they are positions, and/or as they move toward each other. In one example, the facing ends of tubes 201 and 202 may be radiopaque. Such a configuration may provide an operator with an indication of proper positioning and desired movement based on, for example, the orientation (distance, angle, etc.) of the facing ends relative to each other.

B. Magnets

Magnets 204 may be disposed at or near proximal end 216 of distal stent 201. Magnets 206 may be disposed at or near distal end 214 of proximal stent 202. There may be any number of magnets on stents 201 and 202 including, but not limited to, one to ten magnets or eight magnets, as shown in FIG. 2. The number of magnets 204 on stent 201 may be equal to the number of magnets 206 on stent 202 so that, when stents 201 and 202 connect, magnets 204 and 206 may align with each other.

In some examples, magnets 204 and 206 may be affixed onto the ends of wires at the facing ends of stents 201 and 202, e.g., by mechanically fitting the magnets onto the ends of the wires. For example, the magnets may be discs secured within loops formed by or on the ends of the wires, hollow cylindrical bodies (either with or without a tapering outer diameter) crimped about the wires, and/or discs surrounding loops formed by or on the ends of the wires. In some examples, the wires may be formed by a magnetic core surrounded by a coating or covering. Magnets 204 and 206 that are formed by the magnetic core may be embedded in the coating and/or covering over the wires, e.g., magnets 204 and 206 may be embedded within a silicone covering.

In some examples, magnets 204 and 206 may all be disposed on the proximalmost end of stent 201 and the distalmost end of 202. In other examples, magnets 204 and 206 may be staggered, e.g., with some magnets disposed at the proximalmost end of stent 201 and the distalmost end of stent 202, and others set back (e.g., between approximately 0.1 mm and approximately 1 mm) from the proximalmost end of stent 201 and the distalmost end of stent 202. Such staggering, may allow the device to contract down to a smaller diameter, and therefore allow for a smaller delivery system diameter.

Magnets 204 and 206 may include any suitable, known magnets. For example, magnets 204 and 206 may include rare-earth magnets, such as, but not limited to, samarium-cobalt and neodymium magnets. Magnets 204 and 206 may be a sufficient strength to, over time, cause pressure necrosis of the tissue of a stricture by drawing stents 201 and 202 toward each other and against the tissue of the stricture. In some examples, the magnetic pull of magnets 204 and 206 may elongate stent 201 and/or stent 202, reducing radial forces between the elongated stent(s) and tissue surrounding stents 201 and/or 202, thereby facilitating movement of at least one of stents 201 and 202 toward the other.

Magnets 204 and 206 may be any suitable shape, including, for example, circular, elliptical, disc-like, irregular, and/or rectangular. Further, magnets 204 and 206 may have any suitable length, width, and/or thickness. Magnets 204 and 206 may have a length and/or width of between approximately 1 mm and 4 mm, or between approximately 2 mm and approximately 3 mm. Magnets 204 and 206 may have a thickness of between approximately 0.5 mm and 1.5 mm, or of approximately 1 mm.

Magnets 204 and 206 may be configured to have the same orientation of polarities, e.g., the distal-facing sides of magnets 204 and 206 may be N-poles while the proximal-facing sides of magnets 204 and 206 may be S-poles. In such a configuration, the facing ends of medical device 200 will have opposite polarity and thus will be attracted to each other.

Additionally or alternatively, medical device 200 may include one or more tethers (not shown) for tethering stents 201 and 202 to each other. In some examples, these tethers may be elastic. The elastic tethers may help pull stents 201 and 202 together. For example, the elastic tethers may be stretched when pull stents 201 and 202 are placed on both sides of the stricture, with the elastic tethers extending through the stricture. As the elastic tethers return back to a rest state, stents 201 and 202 may be drawn towards each other. In implementations with elastic tethers, no magnets may be required, or weaker magnets may be used, to pull stents 201 and 202 together. It is also contemplated that tethers 180 may link the facing ends of stents 201 and 202.

C. Anti-Migration/Anchoring Features

After stent 201 and 202 are placed on opposite sides of a stricture, the two components may be configured to move closer together due to the attraction between magnetic portions 204 and 206. While the two components move closer together, and/or after they connect, movement of the two components away from the stricture should be avoided to ensure that the drain formed by the two components remains at the stricture. Movement away from the stricture may result in the re-forming of an occlusion at the stricture. In order to prevent medical device 200 from migrating away from the stricture, medical device 200 may incorporate anti-migration and/or anchoring features. Medical device 200 may include any suitable anti-migration and/or anchoring device(s) known in the art. Through incorporation of such anti-migration and/or anchoring features, proximal tube 202 may be configured to have limited or no proximal movement away from the stricture and/or distal tube 202 may be configured to have limited or no distal movement away from the stricture

In one implementation, one or more of stents 201 and 202 may include loop raises 260 and 262. Loop raises 260 and 262 may be formed by one or more struts of stents 201 and 202 that may extend farther out in a radial direction than other struts. Loop raises 260 and 262 may allow tissue ingrowth. Tissue ingrowth may prevent migration of stents 201 and 202 away from the stricture. In some examples, one to ten loop raises 260 and 262 may be included in each of stents 201 and 202. It is also contemplated that one or more of loop raises 260 and 262 may be positioned at any location between the ends 210, 212, 214, and 216 of stents 201 and 202, and/or at the ends 210, 212, 214, and 216. In some examples, stents 201 and 202 may have spikes, prongs, tines, or barbs, 220, 222, and/or 223 similar to anchors 120, 122, and/or 123 of FIG. 1. These spikes 220, 222, and/or 223 may be part of the stent's wires or attached to the stent wires.

It is also contemplated that magnets 204 and 206 may not be flush against the facing ends of medical device 200. In examples in which magnets 204 and/or 206 are not flush against the facing ends of medical device 200, the resulting pockets formed between the facing ends and the magnets 204 and/or 206 may allow tissue ingrowth. In some examples, magnets may be set between approximately 0.5 mm and 4 mm from the facing ends of stents 201 and 202. The tissue ingrowth may limit or prevent migration of the medical device 200. When stents 201 and 202 connect or come close to connecting, the tissue may be trapped between the facing ends and may act as an anchor. In some examples, this anchoring/anti-migration mechanism may be present in tubes 101 and 102 through the trapping of tissue between the facing ends of tubes 101 and 102 arising from attraction between magnetic portions 104 and 106 of FIG. 1.

It should be noted that magnetic portions 104 and 106 described with respect to FIG. 1 and tubes 101 and 102 may be used with medical device 200 of FIG. 2. For example, the ring-shaped magnetic portions 104 and 106 of FIG. 1 may be attached to, or may form, the facing ends (e.g., ends 214 and 216) of stents 201 and 202. The ring-shaped magnetic portions 104 and 106 may be congruent with the facing ends of stents 201 and 202, may encircle stents 201 and 202, or may be slid into the lumens 250 and 252 of stents 201 and 202. Similarly, magnets 204 and 206 described with respect to medical device 200 of FIG. 2 may be used with medical device 100 of FIG. 1. For example, magnets 204 and 206 may be affixed to the facings ends of distal tube 101 and proximal tube 102. These individual magnets 204 and 206 may be attached via adhesive or may be completely or partially embedded into material forming tubes 101 and 102.

III. Pancreatico-Biliary System

FIG. 3 illustrates an exemplary medical drainage device 300 of the present disclosure being positioned in the pancreatico-biliary system. Though use of medical device 300 at a stricture within the pancreatico-biliary system is described below, this disclosure is not limited to such a usage. The medical devices and methods disclosed may be used in any part of a patient's body in order to create a drain through any blockage.

Medical device 300 of FIG. 3 is a two-part drainage system, including an elongate member 301 and an elongate member 302. Elongate members 301 and 302 may each include a distal end, a proximal end, and a lumen extending therebetween. Elongate members 301 and 302 may be, for example, tubes 101 and 102 (see FIG. 1), stents 201 and 202 (see FIG. 2), or a combination of one of tubes 101 and 102 and one of stents 201 and 202. Magnetic portions/magnets 304 and 306 may be disposed at or near the proximal end of elongate member 301 and the distal end of elongate member 302, respectively.

Elongate members 301 and 302 may be positioned in the desired position using an endoscope 360. Endoscope 360 may be inserted into the patient's nose or mouth, through the esophagus and stomach, and into the small intestine 370. A catheter 362 may be pre-loaded with elongate member 301 and elongate member 302, such that catheter 362 may carry elongate members 301 and 302 to the targeted area. Pre-loading may include removably attaching elongate members 301 and 302 to an outer surface of catheter 362, or inserting elongate members 301 and 302 into a lumen within catheter 362. The pre-loaded catheter 362 may be extended out of a distal opening of the endoscope 360 and guided toward the papilla of Vater 334. The papilla of Vater 334 forms the opening where the pancreatic duct 374/376 and the common bile duct empty into the duodenum of the small intestine 370.

FIG. 3 illustrates a pancreatic duct 374/376 with a stricture 380. Stricture 380 may partially or completely block a distal section 374 of pancreatic duct 374/376 from a proximal section 376 of pancreatic duct 374/376. In some aspects, catheter 362 may be advanced from the duodenum of the small intestine 370 through the papilla of Vater 334, and into the body lumen containing stricture 380, e.g., the pancreatic duct 374/376. Catheter 362 may then push elongate member 301 past stricture 380 into distal section 374 of the pancreatic duct 374/376, and deposit elongate member 301 in that position, i.e., distal of stricture 380. Catheter 362 may position elongate member 302 proximal of stricture 380 within proximal section 376 of the pancreatic duct 374/376. The distance elongate members 301 and 302 are placed apart from each other may depend on the size of the stricture and/or the strength of magnetic portions 304 and 306. In some examples, elongate members 301 and 302 may be placed approximately 1 mm to approximately 40 mm apart, or approximately 5 mm to approximately 20 mm apart.

Magnetic portions 304 and 306 may pull elongate members 301 and 302 toward each other. For example, distal elongate member 301 may move proximally and/or proximal elongate member 302 may move distally. Without being bound by theory, it is believed that pressure applied by magnetic portions 304 and 306 and/or the facing ends of elongate members 301 and 302, on tissue at or forming stricture 380, may cause pressure necrosis, effectively “killing” the tissue disposed between elongate members 301 and 302. Thus, elongate members 301 and 302 may “cut’ through the stricture to make a drainage lumen. The drainage lumen may be formed, at least in part, by lumens 350 and 352. In examples in which each elongate member includes multiple magnets, each magnet may line up with another magnet once elongate members 301 and 302 are connected. When elongate members 301 and 302 are connected, their longitudinal axes and lumens 350 and 352 may be aligned or coaxial.

In one example, the amount of time required for elongate members 301 and 302 to connect may depend on a variety of factors, including, but not limited to, the anatomy of the patient, characteristics of stricture 380, the distance elongate members 301 and 302 were initially spaced apart, and/or the strength of magnets 304 and 306. In some examples, it may take approximately 10 days to connect.

In another example, elongate members 301 and 302 may remain separated by tissue pinned between the facing ends of elongate members 301 and 302, and/or extending into openings (not shown) in elongate members 301 and 302 (e.g., openings formed by pockets 130 and 132 of FIG. 1, or apertures in stents 201 and 202 of FIG. 2) due to tissue ingrowth. The drainage lumen may be formed by lumens 350 and 352, and the pinned or ingrown tissue.

The many features and advantages of the disclosure are apparent from the detailed specification, and thus, it is intended by the appended claims to cover such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

Other aspects of the disclosure will be apparent to those skilled in the art from consideration of the specification and practice of the aspects disclosed herein. It is intended that the specification and examples be considered as exemplary only, with a true scope and spirit of the disclosure being indicated by the following claims.

What is claimed is:

1. A method of creating a drain within a lumen of a patient's body, comprising:
positioning a first elongate member distally of a blockage in the lumen, wherein at least one magnetic portion is disposed at or near a proximal end of the first elongate member; and
positioning a second elongate member proximally of the blockage in the lumen, wherein at least one magnetic portion is disposed at or near a distal end of the second elongate member, a gap initially exists between the proximal end of the first elongate member and the distal end of the second elongate member, and attraction between the at least one magnetic portion of the first elongate member and the at least one magnetic portion of the second elongate member facilitates a reduction in the size of the gap.

2. The method of claim 1, further comprising exerting pressure on tissue forming the blockage with the proximal end of the first elongate member and the distal end of the second elongate member via the magnetic attraction.

3. The method of claim 2, further comprising causing pressure necrosis of the tissue via the exerted pressure.

4. The method of claim 1, further comprising forming the drain through connection of the proximal end of the first elongate member with the distal end of the second elongate member.

5. The method of claim 1, wherein each of the first elongate member and the second elongate member includes a tubular plastic member.

6. The method of claim 1, wherein the least one magnetic portion of the first elongate member includes a single ring-shaped magnet.

7. The method of claim 1, wherein the at least one magnetic portion of the first elongate member includes a recess.

8. The method of claim 1, wherein each of the first elongate member and the second elongate member is at least partially formed by a stent.

9. The method of claim 1, wherein the at least one magnetic portion of the first elongate member includes at least eight discrete magnets.

10. A method of creating a drain within a duct of a patient's pancreatico-biliary system, comprising:
positioning a first elongate member distally of a stricture within the duct, wherein at least one magnetic portion is disposed at or near a proximal end of the first elongate member; and
positioning a second elongate member proximally of the stricture within the duct, wherein at least one magnetic portion is disposed at or near a distal end of the second elongate member, the proximal end of the first elongate member is spaced apart from the distal end of the second elongate member, and the at least one magnetic portion of the first elongate member and the at least one magnetic portion of the second elongate member are in close enough of a proximity to each other that magnetic attraction therebetween pulls at least one of the first elongate member and the second elongate member into the stricture within the duct.

11. The method of claim 10, further comprising exerting pressure on tissue forming the stricture with the proximal end of the first elongate member and the distal end of the second elongate member via the magnetic attraction.

12. The method of claim 11, further comprising causing pressure necrosis of the tissue via the exerted pressure.

13. The method of claim 10, further comprising forming the drain through connection of the proximal end of the first elongate member with the distal end of the second elongate member.

14. The method of claim 10, further comprising engaging the duct with at least one anchor extending radially outwardly from an outer surface of at least one of the first elongate member and the second elongate member.

15. The method of claim 10, further comprising elongating at least one of the first elongate member and the second elongate member via the magnetic attraction between the magnetic portions.

16. A medical device for creating a drain within a lumen of a patient's body, comprising:
a first elongate member including a proximal end, a distal end, a lumen extending between the proximal end and the distal end, and a first magnetic portion disposed at or near the proximal end and surrounding the lumen;
a second elongate member including a proximal end, a distal end, a lumen extending between the proximal end and the distal end, and a second magnetic portion disposed at or near the distal end of the second elongate member and surrounding the lumen of the second elongate member, wherein the proximal end of the first elongate member is configured to be drawn toward the distal end of the second elongate member by magnetic attraction between the first and second magnetic portions.

17. The medical device of claim 16, wherein each of the first elongate member and the second elongate member includes a tubular plastic member.

18. The medical device of claim 16, wherein each of the first elongate member and the second elongate member is at least partially constructed of a metal wire stent.

19. The medical device of claim 16, wherein the proximal end of the first elongate member includes at least one opening formed in a surface thereof.

20. The medical device of claim 19, wherein the at least one opening is formed in the proximalmost surface of the proximal end of the first elongate member.
